(19) United States
(12) Patent Application Publication
(10) Pub. No.: US 2006/0093573 A1
(43) Pub. Date: May 4, 2006

(54) Drug composition with antimicrobial activity

(76) Inventor: Apostolos Georgopoulos, Vienna (AT)

Correspondence Address: BAKER & BOTTS, 30 ROCKEFELLER PLAZA, NEW YORK, NY, 10112, US

(21) Appl. No.: 11/230,141

(22) Filed: Sep. 19, 2005

Related U.S. Application Data
(63) Continuation of application No. PCT/AT04/00097, filed on Mar. 17, 2004.

(30) Foreign Application Priority Data
Mar. 20, 2003 (AT) A 453/2003

(51) Int. Cl. A61K 31/785; A61K 31/155

(52) U.S. Cl. 424/78.27; 514/634

(57) ABSTRACT

The invention relates to the use of a polymeric guanidine derivative based on a diamine containing oxyalkylene chains between two amino groups, with the guanidine derivative representing a product of polycondensation between a guanidine acid addition salt and a diamine containing polyalkylene chains between two amino groups, for the production of a drug composition with antimicrobial activity.

### CROSS REFERENCE TO RELATED APPLICATIONS

This application is a continuation of International Patent Application No. PCT/AT2004/000097 filed Mar. 17, 2004, designating the United States and published in the German language on Sep. 30, 2004 under International Patent Publication No. WO 2004/082671, which is based on Austrian Patent Application No. A 453/2003 filed Mar. 20, 2003, to each of which priority is claimed, and each of which is incorporated by reference in its entirety herein.

### FEDERALLY FUNDED GRANT SUPPORT

Not applicable.

### INTRODUCTION

The invention relates to a drug composition with antimicrobial or microbicidal activity, respectively.

### BACKGROUND OF THE INVENTION

Especially in recent years, the development of new antibiotics has been a race against the increasingly developing resistance of microorganisms. The treatment of nosocomial infections, often caused by resistant microorganisms, is one of the major problems encountered by hospitals all over the world. Infections in the field of dermatology such as infections after skin burns, wound infections, decubitus or acne often require lengthy treatments with antibiotics. At the same time, the development of resistances can frequently be observed in staphylococci and pseudomonas strains. Infections of the gastrointestinal tract by Helicobacter pylori also often represent a therapeutic problem. Moreover, especially conjunctivitides, infections of the ENT and genital regions caused by bacteria or viruses may mean therapeutic problems.

### SUMMARY OF THE INVENTION

It is the object of the invention to provide a new drug composition having an increased effectiveness against a plurality of microorganisms.

### DETAILED DESCRIPTION OF THE INVENTION

According to the invention, said object is achieved by using a polymeric guanidine derivative based on a diamine containing oxyalkylene chains between two amino groups, with the guanidine derivative representing a product of polycondensation between a guanidine acid addition salt and a diamine containing polyalkylene chains between two amino groups, as well as the pharmaceutically acceptable salts thereof, for the production of a drug composition with antimicrobial activity.

Furthermore, the invention relates to the use of polyoxyalkylene guanidine salts prepared by using triethylene glycol diamine (relative molecular mass: 148), polyoxypropylene diamine (relative molecular mass: 230) as well as polyoxyethylene diamine (relative molecular mass: 600).

Most preferably poly-[2-(2-ethoxyethoxyethyl)guanidinium hydrochloride] having at least 3 guanidinium groups is contained as the drug substance, with the average molecular mass in particular ranging from 500 to 3.000 D.

The polymeric guanidine derivatives used in accordance with the invention are known from PCT/AT01/00134. By way of reference, the content of said literature is incorporated in the present specification.

The preparation of a preferred representative of the compounds used in accordance with the invention as well as the proof of the antimicrobial activity are described in the following. Exemplary for the class of compounds used in accordance with the invention, the antimicrobial activity of poly-[2-(2-ethoxyethoxyethyl)guanidinium hydrochloride] with an average molecular mass of 1000 D is described hereafter (CAS No. 374572-91-5).

In order to prepare said compound, 4.43 moles of guanidinium hydrochloride were dissolved in 4.03 moles of triethylene glycol diamine at 50° C. Subsequently, this was heated to 120° C. and stirred for two hours at said temperature. Thereafter, said temperature was maintained for 2 hours, then a vacuum (0.1 bar) was applied and stirring under vacuum was continued for two more hours at 170° C. Subsequently, this was aerated to normal pressure, allowed to cool to 120° C. and diluted with demineralized water to approx. 50%. It was neutralized to a pH of approx. 6 with phosphoric acid, allowed to cool and diluted to the desired concentration. The molecular weight was determined to be 1000 D.

The drug substance poly-[2-(2-ethoxyethoxyethyl)guanidinium hydrochloride] exhibits favourable pharmacodynamic properties, along with low toxicity and good tolerance from a pharmacological point of view, and can therefore be used as a medicine in antimicrobial therapy. The drug substance exhibits in particular an excellent antimicrobial activity which could be demonstrated by tests performed on a plurality of microorganisms such as multiresistant bacteria (which are resistant against common antibiotics), fungi (blastomyces, dermatophytes, mould fungi) and viruses such as Herpes simplex. Due to the quick microbicidal activity, a development of resistance is not to be expected, as shown also by tests performed on a comparatively large number of bacterial strains (30 bacterial species and 30 passages).

After an (intravenous or intraperitoneal) systemic administration of poly-[2-(2-ethoxyethoxyethyl)guanidinium hydrochloride] in amounts of up to 15 mg/kg body weight, serum concentrations of up to 100:g/ml are measured in the rat's blood after two hours, whereby, at the same time, the tolerance is good. These concentrations are significantly above the required level to be expected in a therapeutic application. At the same time, good tolerance was observed even with such a high dosage, mortality or serious side-effects did not occur. Therefore, poly-[2-(2-ethoxyethoxyethyl)guanidinium hydrochloride] can be used as an antimicrobial agent.

The drug substance can be administered as a medicine in a suitable drug form alone or together with inorganic or organic pharmacologically indifferent adjuvants. The substance can, for example, be used as a component of an ointment or a solution, wherein the concentration in the ointment may amount to approx. 0.01 to 5 g drug substance per g of ointment or, in the solution, up to a concentration of 15 mg/kg body weight. Due to the above-mentioned favourable pharmacological properties, the application as an ointment or as a solution can be regarded as favourable in a plurality of indications. In particular infections of the skin, mucous membranes, eyes or of the gastrointestinal tract are considered for this purpose. In these areas, the drug substance according to the invention can thus make an important contribution to the avoidance of resistances against antibiotics.

Antimicrobial Activity

In order to assay the antimicrobial activity, 334 microorganisms, clinically relevant isolates and ATCC strains as reference germs were tested for their minimum inhibition concentrations (MIC). Furthermore, the time- and concentration-dependent destruction of germs (killing curves) and, moreover, the potential for the development of resistances was examined.

1. Microorganisms:

In total, 323 bacteria of various species, 10 blastomyces as well as one dermatophyte were used. The tested microorganisms were isolated from the respiratory tract, the urogenital tract, from blood and other biological materials of patients in accordance with standard laboratory methods and were characterized precisely.

2. Test Substance:

The above-described poly-[C2-(2-ethoxy)-ethoxyethyl)-guanidium-chloride] (molecular mass: 1000 D) was used as a stock solution in a 25% aqueous solution. This solution was diluted with sterile water to the respective ready-for-use concentration. The test substances are stored at room temperature.

3. Determination of Minimum Inhibition Concentration (MIC):

The activities of the test substance were examined by the microdilution method in Mueller-Hinton broth according to NCCLS guidelines. The microbial inoculum amounted to at least 5×10⁵ CFU/ml and incubation was carried out for 16 to 20 hours at 36° C./ambient air. The drug substance was used at concentrations of 1000:g/ml to 0.001:g/ml.

The lowest drug substance combination in which no bacterial growth was visible was defined as MIC. In each test batch, ATCC-quality control strains with a known MIC were included.

4. Development of Resistance:

Based on MIC values, 30 bacterial strains were used for experiments on the development of resistance:

Gram-positive germs: MSSA (n=1), MRSA (n=2), MRSE (n=4), VRE (n=5), S. aureus ATCC 29213.

Gram-negative germs: multiresistant E. coli (n=4), Klebsiella pneumoniae (n=1), Klebsiella oxytoca (n=1), Pseudomonas aeruginosa (n=4), multiresistant Pseudomonas aeruginosa (n=4), Acinetobacter sp. (n=2) and E. coli ATCC 35218 and 25922.

All strains were incubated with the test substance at concentrations of 1000:g/ml to 0.001:g/ml for 24 hours. Test tubes in which a growth of bacteria was detected after the first passage were used as inoculum for the second passage. For each bacterial strain, 30 passages were performed, wherein the new MIC was always compared to the MIC from the beginning of the experiments (1^{st} passage).

5. Time- and Concentration-Dependent Destruction of Germs:

For measuring the microbicidal potential of the drug substance, two multiresistant strains of Staphylococci, Enterococci, E. coli and Pseudomonas aeruginosa were used in each case.

The speed of destruction was checked after the addition (time 0) of the bacterial suspension(1×10⁶ to 5×10⁶ CFU/ml) and after 2, 5, 10 and 30 minutes.

In these experiments, a quantitative germ reduction was performed at any time and at any concentration. The drug substance was used at concentrations of 10%, 1%, 0.1% and 0.001%. As a control, a series without drug substance was also always included.

6. Minimum Inhibition Concentration (MIC):

The results of the MIC-test are summarized in Tables 1 to 5.

In case of Staphylococcus aureus and Staphylococcus epidermidis, the drug substance exhibited a very good activity with MIC-values of 4 to 32:g/ml, independently of the resistance profile of the isolates against antibiotics. Multiresistant staphylococci (MRSA) also exhibited MICs in this range (Table 1).

Testing for Enterococcus faecalis also yielded MIC-values of 16 to 32:g/ml (Table 1),wherein multiresistant and vancomycin-resistant enterococci strains (n=5) again did not differ from the sensitive isolates.

Enterobacteriacae: With regard to E. coli, Klebsiella species, Enterobacter species and Proteus mirabilis, the drug substance yielded very good results with MIC-values of 4 to 32:g/ml (Table 2).

The tested Salmonella, Shigella and Yersinia enterocolitica were also seized very well (Table 3).

The nonfermenter group, Pseudomonas aeruginosa and Acinetobacter species, also turned out to be sensitive against the drug substance, with MIC-values of 4 to 32:g/ml (Table 3).

Also in this case, the good effectiveness of the drug substance used according to the invention was shown in five Pseudomonas isolates which were resistant against all clinically relevant antibiotics.

The drug substance was just as effective against Mycobacterium tuberculosis, avium complex, kansaii and gordonae (Table 4) with MIC-values of 16 to 32:g/ml.

Testing of clinically relevant fungus species such as Candida albicans, Cantida tropicalis and Candida parapsilosis (blastomyces) as well as Trichophyton mentagrophytes (dermatophyte) also yielded a very good antimycotic activity (MIC-values of 8 to 32:g/ml) (Table 5).

7. Development of Resistance:

For this purpose, a total of 30 germs from nine different pathogenic bacterial species—Gram-positive germs such as Staphylococcus aureus and epidermidis as well as Gram-negative germs such as E. coli, Klebsiella spp., Pseudomonas aeruginosa and Acinetobacter spp.—were used. Both sensitive ATCC-strains and multiresistant clinical isolates were tested.

After 30 passages, no development of resistance could be observed in both groups, i.e. no increase in MIC-values was detected.

8. Time- and Concentration-Dependent Destruction of Germs:

In order to evaluate the bactericidal property of the drug substance, the speed of the destruction of bacteria was determined.

For the experiments, two strains of Staphylococcus aureus, Enterococcus faecalis, E. coli and Pseudomonas aeruginosa were used in each case.

In case of Staphylococcus aureus, E. coli and Pseudomonas aeruginosa, the power of destruction of the drug substance persisted up to a concentration of 0.01%, immediately after the addition of the inoculum (0 to 30 seconds).

In case of enterococci, a time period of 10 to 30 minutes was necessary for the destruction of germs, with a concentration of 0.01% drug substance.

### WORKING EXAMPLES

Preferred embodiments of the invention are explained in more detail by way of the following examples.

TABLE 1
Effectiveness against Gram-positive bacteria

| | Number of strains n (%) | | | |
|---|---|---|---|---|
| | having a MIC (in mg/l) of | | | |
| Species | 4 | 8 | 16 | 32 |
| MSSA | 0 (0) | 6 (17.1) | 20 (57.1) | 9 (25.8) |
| (n = 35) | | | | |
| MRSA | 3 (5) | 10 (16.7) | 38 (63.3) | 9 (15) |
| (n = 60) | | | | |
| Enterococcus faecalis | 0 (0) | 0 (0) | 16 (50) | 16 (50) |
| (n = 32) | | | | |

TABLE 2
Effectiveness against Enterobacteriacae

| | Number of strains n (%) | | | |
|---|---|---|---|---|
| | having a MIC (in mg/l) of | | | |
| Species | 4 | 8 | 16 | 32 |
| Klebsiella spp. | 0 (0) | 5 (11.6) | 18 (41.9) | 20 (46.5) |
| (n = 43) | | | | |
| Escherichia coli | 2 (3.4) | 17 (28.8) | 39 (66.1) | 1 (1.7) |
| (n = 59) | | | | |
| Enterobacter spp. | 1 (5.9) | 1 (5.9) | 12 (70.6) | 3 (17.6) |
| (n = 17) | | | | |
| Proteus spp. | 0 (0) | 0 (0) | 3 (42.9) | 4 (57.1) |
| (n = 7) | | | | |

TABLE 3
Effectiveness against Gram-negative bacteria

| | Number of strains n (%) | | | |
|---|---|---|---|---|
| | having a MIC (in mg/l) of | | | |
| Species | 4 | 8 | 16 | 32 |
| Pseudomonas spp. | 1 (1.8) | 5 (9.1) | 32 (58.2) | 17 (30.9) |
| (n = 55) | | | | |
| Acinetobacter spp. | 0 (na) | 0 (na) | 1 (na) | 1 (na) |
| (n = 2) | | | | |
| Salmonella spp. | 0 (na) | 2 (na) | 4 (na) | 0 (na) |
| (n = 6) | | | | |
| Shigella spp. | 0 (na) | 0 (na) | 1 (na) | 1 (na) |
| (n = 2) | | | | |
| Yersinia enterocolitiea | 0 (na) | 0 (na) | 0 (na) | 1 (na) |
| (n = 1) | | | | |

TABLE 4
Effectiveness against Mycobacteria

Number of strains n (%)
having a MIC (in mg/l) of

| Species | 4 | 8 | 16 | 32 |
|---|---|---|---|---|
| Mycobacterium spp. | 0 (na) | 0 (na) | 5 (na) | 1 (na) |
| (n = 6) | | | | |

TABLE 5
Effectiveness against fungi

Number of strains n (%)
having a MIC (in mg/l) of

| Species | 4 | 8 | 16 | 32 |
|---|---|---|---|---|
| Candida spp. | 0 (na) | 1 (na) | 3 (na) | 6 (na) |
| (n = 10) | | | | |
| Trichophyton | 0 (na) | 0 (na) | 1 (na) | 0 (na) |
| mentagrophytes (n = 1) | | | | |

Clinical Activity

In order to confirm the good in-vitro activity of the drug substance in vivo, the substance was used under clinical conditions on voluntary patients suffering from problematic infections. The substance was used in an aqueous solution or in gel form at a concentration of 0.5%.

Patients

12 patients aged from 35 to 62 years were treated with the drug substance.

2 patients suffering from chronic gastritis (Helicobacter pylori positive)

3 patients suffering from chronic inflammations of gingiva and oral mucosa

3 patients suffering from chronic inflammation of vaginal mucosa

1 patient suffering from foot gangrene

3 patients suffering from onichomycosis (1 patient suffering from hand mycosis, 2 patients suffering from foot mycosis)

Results

The clinical effectiveness of the drug substance in 12 selected patients is summarized in Table 6. Type of treatment, duration of treatment, principal clinical signs and clinical success can be seen as well.

In short, a convincing clinical effectiveness could be observed in all 12 cases after a treatment period of 2 to 28 days. A clear improvement of principal signs occurred, whereas local tolerance was excellent. In none of the cases, side-effects such as reddening, irritations or nausea occurred.

TABLE 6

| | | | | Duration of | |
|---|---|---|---|---|---|
| Number of | | Clinical | Type of | treatment | Clinical |
| patients | Indication | symptoms | treatment | (in days) | success |
| 2 | chronic gastritis | gastric | 0.5% solution | 14 | no complaints |
| | (H. pylori | tenderness on | orally, twice | | |
| | positive) | pressure, | a day | | |
| | | heartburn | | | |
| 3 | inflammations | painful centres | 0.5% solution | 2 | significant |
| | of gingiva and | of | locally, twice | | healing, no |
| | oral mucosa | inflammation, | a day | | pain, no |
| | | bleeding from | | | bleeding from |
| | | the gums | | | the gums |
| 3 | chronic | burning, | 0.5% solution | 5 | no complaints |
| | inflammation of | discharge, | locally, twice | | |
| | vaginal mucosa | itchiness | a day | | |
| 1 | foot gangrene | ulcerous | 0.5% solution | 28 | clear |
| | | changes, | locally, three | | improvement |
| | | oedematous | times a day | | of local |
| | | penetration, | | | findings, no |
| | | bad smell, | | | development |
| | | surgery | | | of smell, start |
| | | contraindicated | | | of surgical |
| | | | | | preparations |
| 3 | hand and foot | itchiness, | 0.5% solution | 28 | no itchiness, |
| | mycosis | deformation | locally, three | | regrowth of |
| | | and | times a day | | healthy nail |
| | | discolouration | | | |
| | | of nail | | | |

1. A method for producing an antimicrobial effect in a subject, comprising administering to the subject, an effective amount of a polymeric guanidine derivative based on a diamine containing oxyalkylene chains between two amino groups, with the guanidine derivative representing a product of polycondensation between a guanidine acid addition salt and a diamine containing polyalkylene chains between two amino groups.

2. The method of claim 1, wherein the family of polyoxyalkylene guanidine salts, include triethylene glycol diamine (relative molecular mass: 148), polyoxypropylene diamine (relative molecular mass: 230) and polyoxyethylene diamine (relative molecular mass: 600).

3. The method of claim 1, wherein poly-[2-(2-ethoxyethoxyethyl)guanidinium hydrochloride] having at least 3 guanidinium groups is used.

4. The method of claim 3, wherein the average molecular mass of the drug substance is from 500 to 3,000.

5. The method of claim 1, wherein the drug composition is designed as a drug composition for human or veterinary use.

6. The method of claim 2, wherein the drug composition is designed as a drug composition for human or veterinary use.

7. The method of claim 3, wherein the drug composition is designed as a drug composition for human or veterinary use.

8. The method of claim 4, wherein the drug composition is designed as a drug composition for human or veterinary use.

9. A method of treating infection in a subject comprising administering to the subject, a therapeutically effective amount of a polymeric guanidine derivative based on a diamine containing oxyalkylene chains between two amino groups, with the guanidine derivative representing a product of polycondensation between a guanidine acid addition salt and a diamine containing polyalkylene chains between two amino groups.

10. The method of claim 9, wherein the polymeric guanidine derivative is administered as a formulation selected from the group consisting of an intravenous, intraperitoneal, ointment or solution preparation.

11. The method of claim 9, wherein the family of polyoxyalkylene guanidine salts, include triethylene glycol diamine (relative molecular mass: 148), polyoxypropylene diamine (relative molecular mass: 230) and polyoxyethylene diamine (relative molecular mass: 600).

12. The method of claim 9, wherein poly-[2-(2-ethoxyethoxyethyl)guanidinium hydrochloride] having at least 3 guanidinium groups is used.

13. The method of claim 9, wherein the average molecular mass of the polymeric guanidine derivative is from 500 to 3,000.
